(19) United States
(12) Patent Application Publication
(10) Pub. No.: US 2025/0043258 A1
(43) Pub. Date: Feb. 6, 2025

(54) CRISPR-based DNA Repair-Inhibiting Intratumoral Cancer Therapy

(71) Applicants: Sarfaraz K. Niazi, Deerfield, IL (US); Matthias Magoola, Kampala (UG)

(72) Inventors: Sarfaraz K. Niazi, Deerfield, IL (US); Matthias Magoola, Kampala (UG)

(73) Assignee: RNA Therapeutics, Inc., Deerfield, IL (US)

(21) Appl. No.: 18/496,845

(22) Filed: Oct. 28, 2023

(51) Int. Cl. C12N 9/22; A61K 31/502; A61K 31/519; A61K 31/522; A61K 31/5386; A61K 31/551; A61K 31/7088; A61K 38/46; C12N 15/11; C12N 15/88

(52) U.S. Cl. CPC C12N 9/22; A61K 31/502; A61K 31/519; A61K 31/522; A61K 31/5386; A61K 31/551; A61K 31/7088; A61K 38/465; C12N 15/11; C12N 15/88; C12N 2310/20

(57) ABSTRACT

A novel composition for treating cancer comprising of a gRNA-Cas9 complex capable of locating, binding, and causing a double-strand break of the gene that is prevented from repair by accompanying DNA repair inhibitors.

### SEQUENCE LISTING

The instant application contains a Sequence Listing, which has been submitted electronically in ASCII format and is hereby incorporated by reference in its entirety. Said ASCII text file, created on Sep. 20, 2024, is named CRISPR.xml, and is 7 kb in size.

### BACKGROUND OF THE INVENTION

Cancer is the second leading cause of death on the planet today, a disease that affects more than 32 million people globally and is predicted to grow by as much as 70% in the next 20 years. Over the centuries, essential discoveries identified the biological and pathological features of tumors without any practical therapeutic approaches until the end of the 1800s, when the discovery of X-rays, and later after the Second World War, came the discovery of cytotoxic antitumor drugs for the treatment of various hematological and solid tumors. The next breakthrough took place at the beginning of the '80s, with the arrival of molecular and cellular biology allowing the development of specific drugs for some molecular targets involved in neoplastic processes, giving rise to targeted therapy.

Research is now focused on the development of cell therapies, anti-tumor vaccines, and new biotechnological drugs such as the 14 Antibody-drug conjugates (ADCs) products, including an immunotoxin Lumoxiti (moxetumomab pasudotox-tdfk) currently approved. However, all these therapies have the potential for side effects, the off-target impact affecting healthy cells that are not the target of the delivery systems.

Gene mutations play a significant role in the development of cancer. Numerous genes can undergo mutations, leading to dysregulated cell growth, impaired DNA repair mechanisms, and other hallmarks of cancer. Genes can be mutated in several different ways. The simplest type of mutation is a change in the DNA that makes up the gene. DNA is made up of 4 other bases arranged in a specific order. A mutation happens when a base is changed, or the order of the bases is changed. Sometimes, gene mutations can also change the structure of an entire chromosome.

An oncogene is a gene that has the potential to cause cancer. These genes are often mutated or expressed at high levels in tumor cells. Most normal cells will undergo a programmed form of rapid cell death (apoptosis) when critical functions are altered and malfunctioning. Activated oncogenes can cause those cells designated for apoptosis to survive and proliferate instead. Most oncogenes began as proto-oncogenes, normal genes involved in cell growth and proliferation or inhibition of apoptosis. If, through mutation, normal genes promoting cellular growth are up-regulated (gain-of-function mutation), they will predispose the cell to cancer; thus, they are termed “oncogenes.” Usually, multiple oncogenes and mutated apoptotic or tumor suppressor genes will all act in concert to cause cancer. Since the 1970s, dozens of oncogenes have been identified in human cancer. Many cancer drugs target the proteins encoded by oncogenes.

In addition to single-nucleotide mutations, most cancer cells contain somatic mutations, primarily small insertions, and deletions that do not exist in neighboring normal cells. Whole-exome sequencing of solid tumors revealed that ˜5% of the total somatic variation in cancer cells is like this, thus suggesting that these can also be candidates for tumor-specific neoantigen immunotherapy. A more recent study that analyzed 2,658 whole-cancer genomes reported that most tumors had 100 to 1,000 Insertion-deletions. Incorrect DNA repairs by nonhomologous end joining (NHEJ) or microhomology-mediated end joining (MMEJ) might cause Insertion-deletion formation in proliferating tumors. Recently, other DNA repair pathways, including transcription-coupled repair and mismatch repair pathways, were also suggested, contributing to insertion-deletion formation in different cancer cells.

Examples of common mutations include (Gene-UniProt Code) ABL1-P00519; AKT1-P31749; AKT2-P31751; AKT3-Q9Y243; ALK-Q9UM73; APC-096017; AR-P10275; ARID1A-014497; ATM-Q13315; ATR-Q13535; B2M-P61769; BAP1-Q92560; BARD1-Q99728; BCL10-Q92874; BCL2-P10415; BCL2L1-Q07817; BCL6-P41182; BCR-P11274; BIRC3-Q13485; BLK-P51451; BMPR1A-P36894; BRAF-P15056; BRCA1-P38398; BRCA2-P51587; BRIP1-Q9BX63; BTG1-P62324; CALR-P27797; CARD11-Q9BVK6; CASP8-Q14790; CBL-P22681; CCND1-P24385; CCND2-P30279; CCND3-P30281; CD274-Q9NZQ7; CD276-Q9BQ51; CD79A-P11912; CD79B-P40259; CDH1-P12830; CDH2-P19022; CDK4-P11802; CDK6-Q00534; CDKN1A-P38936; CDKN1B-P46527; CDKN2A-P42771; CDKN2B-Q99816; CDKN2C-Q9NRY4; CEBPA-P49715; CHEK1-O14757; CHEK2-O96017; CREBBP-Q92793; CRLF2-Q9GZX6; CSF1R-P07333; CTNNB1-P35222; CUL3-Q13618; CYLD-Q9NQC7; DAXX-Q9UER7; DDR2-Q16832; DICER1-Q9UPY3; DNMT3A-Q9Y6K1; EGFR-P00533; EML4-Q6PML9; EP300-Q09472; ERBB2-P04626; ERBB3-P21860; ERBB4-Q15303; ESR1-P03372; ETV1-P50549; ETV4-P43268; ETV5-Q00534; ETV6-P41212; EZH2-Q15910; FANCA-O15360; FANCD2-Q9BXW9; FANCE-Q9HB96; FANCF-Q9NPI8; FANCG-015287; FAS-P25445; FBXW7-Q969H0; FGFR1-P11362; FGFR2-P21802; FGFR3-P22607; FLT3-P36888; FOXP1-Q9H334; GATA1-P15976; GATA2-P23769; GATA3-P23771; GNA11-P29992; GNAQ-P50148; GNAS-095467; GNB1-P62873; GNB2L1-P62873; GPR124-Q86YT4; GRIN2A-Q12879; H3F3A-P84243; H3F3B-P84244; HIF1A-Q16665; HRAS-P01112; IDH1-O75874; IDH2-O75874; IDH3A-O43837; IDH3B-Q9Y6K9; IDH3G-O75390; IGF1R-P08069; IL7R-P16871; INSR-P06213; JAK1-P23458; JAK2-060674; JAK3-P52333; KDM5A-Q9UGL1; KDM6A-015550; KDR-P35968; KEAP1-Q14145; KIT-P10721; KMT2A-Q03164; KMT2C-O14686; KMT2D-014686; KRAS-P01116; LIFR-P42702; LRP1B-Q6UWL6; MAP2K1-Q02750; MAP2K2-P36507; MAP2K4-P45985; MAP3K1-Q13233; MAP3K4-Q9Y4K4; MAPK1-P28482; MAPK3-P27361; MAX-P61244; MCL1-Q07820; MDM2-Q00987; MED12-095243; MEN1-000255; MET-P08581; MLH1-P40692; MPL-P40238; MSH2-P43246; MSH6-P52701; MTOR-P42345; MYB-P10242; MYC-P01106; MYCL-P12524; MYCN-P04198; MYD88-Q99836; NCOA1-Q15788; NCOA2-Q15596; NCOA3-Q9Y6Q9; NF1-P21359; NF2-P35240; NOTCH1-P46531; NOTCH2-Q04721; NPM1-P06748; NRAS-P01111; NSD1-Q96L73; NTRK1-P04629; NTRK2-Q16620; NTRK3-Q16288; PAK1-Q13153; PAX3-P23760; PAX7-P23771; PBRM1-Q86U86; PDGFRA-P16234; PDGFRB-P09619; PHF6-Q8IXJ9; PIK3CA-P42336; PIK3CB-P42338; PIK3CD-000329; PIK3CG-P48736; PIK3R1-P27986; PIK3R2-O00459; PIK3R3-O75771; PLCB1-Q01970; PMS2-P54278; POLD1-P28340; POLH-Q9Y253; PPM1D-015297; PPP2R1A-P30153; PPP2RIB-Q13362; PRDM1-O75626; PRKACA-P17612; PRKCA-P17252; PRKCB-P05771; PRKCD-Q05655; PRKDC-P78527; PTCH1-Q13635; PTEN-P60484; PTPN11-Q06124; RAD21-O60216; RAD50-Q92878; RAD51-Q06609; RAF1-P04049; RB1-P06400; RET-P07949; RHOA-P61586; RNF43-Q68DV7; ROS1-Q9NRD0; RUNX1-Q01196; SDHA-P31040; SDHB-P21912; SDHC-Q99643; SDHD-014521; SETBP1-Q9Y4C1; SF3B1-Q15788; SIRT2-Q8IXJ6; SLX4-Q8IY92; SMAD2-Q15796; SMAD3-P84022; SMAD4-Q13485; SMC1A-Q14683; SMC3-Q9NTJ3; SMO-Q99835; SPOP-O43791; SRC-P12931; SRSF2-P62807; STAG2-Q8N8T8; STAT3-P40763; STAT5B-P51692; STK11-Q15831; SUZ12-Q15022; SYNE1-Q8NF91; TBL1XR1-Q9UBK7; TCF7L2-Q9NQB0; TERT-014746; TET2-Q6N021; TFEB-P19484; TGFBR2-P37173; TLR4-O00206; TNFAIP3-Q6ZNK6; TP53-P04637; TPMT-P51580; TRAF3-O43184; TRIM24-Q13263; TRIM33-015164; TSC1-Q92574; TSC2-P49815; U2AF1-P26368; UBR5-Q7Z6Z7; USP9X-Q93008; VHL-P40337; WHSC1-060814; WRN-Q14191; WT1-P19544; XPO1-014980; YAP1-P46937; YES1-P07947; ZBTB16-P41182; ZEB1-P37275; ZFHX3-Q15911; ZMYM2-Q13263; ZMYM3-Q9Y6X9; ZMYM5-Q9UHG3; ZNF217-P14373; and ZNF750-Q6ZMV9.

Identifying the most common gene mutations leading to cancer is a complex task, as it can vary depending on the cancer type and population studied. However, there are some well-established and frequently observed gene mutations in cancer. The Cancer Genome Atlas (TCGA) project is a comprehensive effort to analyze the genomic landscapes of various cancer types, profiling thousands of tumor samples from different cancer types, identifying recurrent mutations in specific genes, and providing the frequency and distribution of gene mutations across multiple cancer types,

Targeted therapy, a more recent advancement in cancer treatment, focuses on specific molecular targets in cancer cells. This approach aims to inhibit the growth and spread of cancer while minimizing damage to normal cells. Targeted therapies have shown significant success in treating certain types of cancer, including breast cancer, lung cancer, and leukemia.

Significant challenges for mAb-based drug delivery include the identification of target antigens that are overexpressed specifically on the tumor surface, the circumvention of potential immunogenicity of the complexes, and the determination of the right size of the conjugates that can penetrate tumors, yet not to be cleared by kidneys too fast. In recent years, significant advancements have been made in the development and clinical application of ADCs, enhancing their effectiveness, and expanding their therapeutic potential.

Antibody-drug conjugates (ADCs) are targeted therapy that combines the specificity of monoclonal antibodies (mAbs) with the cytotoxic properties of small-molecule drugs. The ADCs also damage the DNA in neighboring healthy cells, which then causes undesirable side effects, including cell death and mutations.

### BRIEF SUMMARY OF THE INVENTION

An ideal cancer therapeutic strategy involves the selective killing of cancer cells without affecting the surrounding normal cells. However, researchers have failed to develop compositions for achieving cancer cell death because of shared features between cancerous and normal cells. In this invention, we have developed a therapeutic strategy to selectively induce cancer cell death by targeting mutated genes by sgRNA complexed with Cas9 protein to cause a double-strand break of the gene. The break is prevented from repair by accompanying PRP inhibitors to prevent homolog repair and PK-GS inhibitors to prevent non-homologous repair, resulting in cell destruction. There are a few dual inhibitors:

| Dual Inhibitor Name | Chemical Formula | CAS NumberG12 |
|---|---|---|
| AZD7648 | C19H22N4O2 | CAS: 2101156-99-5 |
| E7449 | C25H21F2N3O2 | CAS: 1038915-60-4 |
| KU-0058948 | C22H27N5O3 | CAS: 1313364-08-1 |
| NU5455 | C20H20N8 | - CAS: 2212441-05-7 |
| NU7441 | C21H20N6O | CAS: 503468-95-9 |
| Olaparib (when used at | C24H23FN4O3 | CAS: 763113-22-0 |
| higher concentrations) | | |

Guide RNAs (gRNAs) are essential components of the CRISPR/Cas9 genome editing system, designed to target specific DNA sequences. When aiming to edit a mutated gene, the exact sequence of the mutation, typically 20 nucleotides in length, is first identified. The Cas9 protein from Streptococcus pyogenes must recognize a specific protospacer adjacent motif (PAM) sequence, “NGG,” which must be immediately adjacent to this 20-nucleotide target. Therefore, the complete target site in the genome appears as a sequence like 5′-ACGTACGTACGTACGTACGT-NGG-3′ (SEQUENCE NO. 1). The gRNA is then constructed to complement this 20-nucleotide target sequence and is combined with a constant scaffold sequence that allows it to function with the Cas9 protein. Designing gRNAs requires meticulous attention due to factors like GC content and potential off-target effects. Many tools are available online to help ensure the efficiency and specificity of the gRNA design.

BRCA1 and BRCA2 are essential genes involved in DNA repair, and mutations in them are linked to an elevated risk of breast and ovarian cancers. However, not every mutation in these genes necessarily increases cancer susceptibility. The mutations are categorized into pathogenic mutations, which have a confirmed association with increased cancer risk, often producing a non-functional protein; benign mutations, which don't increase cancer risk; and Variants of Uncertain Significance (VUS), whose clinical impact is yet to be determined. These mutations can manifest in several ways, from single DNA base pair changes (missense mutations) to introducing a premature stop signal (nonsense mutations). Some mutations may even change the gene's reading frame (frameshift mutations) or result in the deletion or duplication of large gene segments. Beyond BRCA1 and BRCA2, other genes like PALB2, PTEN, TP53, and CHEK2, among others, also carry mutations associated with increased breast cancer risk.

Nonetheless, having such a mutation doesn't guarantee the development of breast cancer; it merely indicates a heightened risk. Other factors, including environment, lifestyle, and additional genetic elements, come into play. It's also noteworthy that while BRCA mutations are well-documented, most breast cancer cases are sporadic and don't present a clear hereditary connection. For those concerned about their genetic predisposition, seeking genetic counseling and testing can offer more tailored insights.

The KRAS gene, pivotal in cell signaling and growth, has been identified as a hotspot for mutations in various cancers. When mutated, KRAS can lead to the unchecked growth and proliferation of cells, a hallmark of cancer development. These mutations can be broadly classified: pathogenic mutations, which are known to increase cancer risk by leading to an active or constitutively signaling protein; benign mutations, which don't elevate cancer risk; and Variants of Uncertain Significance (VUS), for which the clinical implications remain undefined. The nature of these mutations can vary, from single base pair alterations (missense mutations) that change one amino acid in the protein to less common insertions or deletions. Notably, specific mutations in the KRAS gene, especially at codons 12 and 13, are prevalent in certain colorectal, lung, and pancreatic cancers. The KRAS gene, pivotal in cellular signaling, is notorious for its role in cancer when mutated. One specific mutation that has garnered significant attention is the G12C mutation. This mutation is localized at codon 12 of the KRAS gene, where the original glycine (G) encoded by the codon GGT is replaced by cysteine (C), resulting in the codon TGT. This seemingly small sequence alteration can lead to significant deregulation in cell growth, making it a hot spot for oncogenic activity. Human KRAS protein is a small guanosine triphosphatase (GTPase) component of the mitogen-activated protein kinase (MAPK) signaling pathway. High occurrences of KRAS mutations in some types of cancers make KRAS one of the most important targets in oncology for drug development. The four most frequent KRAS mutations are G12D, G12V, G13D, and G12C. Notably, the G12C mutation is among the most prevalent KRAS mutations in lung adenocarcinoma and has been a prime target in recent therapeutic research endeavors. The G12C sequence is:

(SEQUENCE NO. 2)
MTEYKLVVVGACGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKC.

CRISPR-Cas9 is a revolutionary gene-editing technology that holds significant promise for cancer treatment. While the application of CRISPR-Cas9 in cancer therapy is still in its early stages of development, ongoing research is exploring its potential. The current research focus is the application of CRISPR-Cas9 in cancer research and therapeutics, including functional genomics, identification of cancer drivers, and development of novel therapeutic strategies. Adverse effects associated with CRISPR-Cas9 gene editing have been a subject of investigation. The potential risks and challenges associated with off-target mutations induced by CRISPR-Cas9, including unintended alterations in genomic sequences, require developing strategies and techniques to minimize off-target effects and enhance the specificity of CRISPR-Cas9 editing.

### DETAILED DESCRIPTION OF THE INVENTION

Guide RNAs (gRNAs) are integral to the CRISPR genome editing system, and their types and structures vary depending on the specific CRISPR system in use. For the widely used Streptococcus pyogenes Cas9 system, the gRNA often fuses two components: the crRNA (CRISPR RNA) and the tracrRNA (trans-activating crRNA), forming a single molecule known as sgRNA. This molecule features a 20-nucleotide target-specific sequence trailed by a consistent scaffold sequence. In contrast, the CRISPR-Cpf1 (or Cas12a) system demands a shorter and simpler gRNA devoid of the tracrRNA part. The CRISPR-Cas13 system, which uniquely targets RNA rather than DNA, employs a gRNA designed to complement the target RNA sequence without the tracrRNA component. Specialized applications might use dual gRNAs, guiding the Cas protein to two separate genomic locations, or self-limiting gRNAs with built-in temporal control. MS2-tagged gRNAs, which incorporate an MS2 RNA motif, allow for the recruitment of MS2-fused proteins to DNA binding or cleavage sites. Meanwhile, synthetic gRNAs, crafted chemically, might carry modified bases or backbones for enhanced stability or function. Despite the variations, all gRNAs share a central purpose: guiding the Cas protein to a designated location within the genome or transcriptome.

Guide RNA (gRNA) can be complexed with a Cas9 protein wherein cytotoxic payload using different conjugation strategies. One of the most common approaches is using a bifunctional linker that can covalently attach the gRNA and the cytotoxic molecule. The linker is designed in such a way that it can react with specific functional groups present on the gRNA and the cytotoxic molecule.

The most optimized sequence of guide RNA (gRNA) to attach to a mutated oncogene would depend on the specific sequence of the oncogene mutation. The gRNA is a short RNA sequence that guides the CRISPR-Cas system to the target site on the DNA.

Once the target mutation and its sequence context are determined, bioinformatics tools such as CRISPR Design or E-CRISP can be used to design the gRNA sequence. The designed gRNA can then be cloned into a vector system with the Cas protein and delivered into the cells for targeted editing.

To design an optimized gRNA sequence for a specific oncogene mutation, several factors are considered:

Specificity: The gRNA should be highly specific to the target mutation and not bind to other genomic regions to avoid off-target effects.

Efficiency: The gRNA should efficiently target the mutated oncogene and induce a high level of editing.

Accessibility: The target site should be accessible to the CRISPR-Cas system.

PAM (Protospacer Adjacent Motif) site: The target site should have a PAM sequence adjacent for the Cas protein to bind and cleave.

Structural constraints: The target site should be in a region of the DNA where the Cas protein can access and cleave the DNA.

Designing a crRNA for a specific mutation, such as the G12C mutation, starts by pinpointing the exact location and associated DNA sequence. For the G12C mutation, which signifies a transition from a glycine (G) to a cysteine (C) at the 12th amino acid position, it's crucial to identify the precise nucleotide alteration that results in this amino acid swap. Using a hypothetical example, the codon for glycine could change from GGT to TGT. From there, a 20-nucleotide sequence surrounding this mutation is chosen, ideally encompassing 10 nucleotides on either side of the mutation. This sequence is selected to ensure specificity so the Cas9 protein won't mistakenly target other genes. An essential aspect of the CRISPR-Cas9 system from Streptococcus pyogenes is the Protospacer Adjacent Motif (PAM) sequence, “NGG.” This sequence must be present immediately after the 20-nucleotide target sequence in the DNA, as Cas9 requires this for binding and activity. Once the DNA target sequence is identified, the complementary RNA sequence is generated to form the crRNA. As a final step, testing the designed crRNA in a controlled environment, like a cell culture, is imperative to ensure its efficiency and specificity. Moreover, many researchers lean on online tools and platforms that offer algorithms for optimal crRNA design, considering factors like potential off-target effects and sequence uniqueness.

Guide RNA (gRNA) is used in the CRISPR-Cas9 genome editing system. The gRNA directs the Cas9 protein to a specific location in the DNA where a cut or modification needs to be made. The gRNA consists of two main parts:

CRISPR RNA (crRNA): This sequence is typically 20 nucleotides long and is complementary to the target DNA sequence you wish to modify or cut. Its sequence is what provides the specificity to the system. The actual sequence of this part is variable and depends on the desired target DNA site.

Trans-activating CRISPR RNA (tracrRNA): This is a constant sequence that binds to the Cas9 protein and facilitates the interaction of Cas9 with the DNA. Its sequence is derived from the bacterial system where CRISPR-Cas9 originated and remains consistent across different experiments.

The two RNA sequences, crRNA and tracrRNA, can be combined into a single synthetic molecule called the single guide RNA (sgRNA). The sgRNA streamlines the CRISPR-Cas9 system by consolidating the two RNA components.

The exact sequence of the tracrRNA part (for Streptococcus pyogenes Cas9) is:

(SEQUENCE NO. 3)
3′-GUUUUAGAGCUAGAAAUAGCAAGUUAAAAUAAGGCUAGUCCGUUAUC
AACUUGAAAAAGUGGCACCGAGUCGGUGCUUUUUU-5′.

The crRNA sequence will be variable depending on the DNA target site. One would design a specific 20 nucleotide sequence to match the desired target in the genome, and this would be attached to the consistent tracrRNA sequence in the case of a sgRNA design.

Binding guide RNA (gRNA), Cas9, and DNA repair inhibitors to specifically target mutated cells, such as cancer cells, present an intriguing but complex therapeutic strategy. The first step involves complexing gRNA with the Cas9 protein to form a ribonucleoprotein (RNP) complex, which targets and introduces DNA breaks at specific genomic locations. While DNA repair inhibitors, typically small molecules, cannot be directly bound to the Cas9 protein, they can be encapsulated within a delivery vehicle carrying the Cas9-gRNA complex. Potential delivery mechanisms include lipid nanoparticles (LNPs), viral vectors, and exosomes. LNPs can be functionalized with ligands or antibodies to recognize markers unique to cancer cells for enhanced specificity. Once inside the cell, the Cas9-gRNA complex targets the mutated DNA sequence, and the DNA repair inhibitor impedes the cell's ability to fix the induced DNA damage, culminating in cell death. However, the challenge lies in ensuring specificity to prevent off-target effects and unintentional harm to healthy cells. Creating a combined therapeutic approach would necessitate extensive research, optimization, and rigorous testing.

As an example, the G12C mutation is a specific point mutation commonly found in the KRAS gene, particularly in certain cancers. The G12C mutation indicates that the 12th amino acid, glycine (G), has been replaced with cysteine (C). However, to specify a “20 nucleotide target sequence,” we′d need the exact nucleotide change responsible for this amino acid switch and some surrounding context. The G12C mutation in KRAS is typically due to a change from GGT (glycine) to TGT (cysteine) at the codon position 12. For instance, if we have a longer sequence of KRAS: . . . . CTGGTGGCGTAGGCAAGAGTGCCTTGACGATACAGCTA (SEQUENCE NO. 4) . . . where GGT is the wild-type codon for glycine, the G12C mutation would change it to: . . . . CTGGTGGCGTAGGCAAGTGTGCCTTGACGATACAGCTA (SEQUENCE NO. 5) . . . (Note the change from GGT to TGT.) From this, a 20-nucleotide target sequence surrounding the mutation could be: . . . CGTAGGCAAGTGTGCCTTGA (SEQUENCE NO. 6) . . . .

DNA repair inhibitors represent a promising class of drugs, especially in cancer therapy. Some of the most notable DNA repair inhibitors include Poly (ADP-ribose) polymerase (PARP) inhibitors, such as olaparib, rucaparib, and niraparib, which hinder the base excision repair (BER) pathway. Other inhibitors target kinases involved in DNA damage detection and response, such as ATM (Ataxia-telangiectasia mutated) and ATR (ATM and Rad3-related) inhibitors. DNA-dependent protein kinase (DNA-PK) inhibitors, which interfere with the non-homologous end joining (NHEJ) pathway, represent another category. Additionally, checkpoint kinase inhibitors against proteins like CHK1 and CHK2 disrupt cell cycle checkpoints, potentially preventing cells from repairing DNA damage before cell division. While binding these inhibitors directly to the gRNA-Cas9 complex is intriguing, it poses challenges. These inhibitors are more likely to be co-delivered in a shared delivery system, such as lipid nanoparticles, rather than being directly bound to the gRNA-Cas9 complex.

Delivering the gRNA-Cas9 complex efficiently to target cells is pivotal for successful CRISPR-Cas9 therapies. A promising composition involves using lipid nanoparticles (LNPs), lipid-based vesicles capable of encapsulating nucleic acids or protein complexes. Within these LNPs, the gRNA and Cas9 can be encapsulated as a combined ribonucleoprotein (RNP) complex or as separate entities. When Cas9 is introduced as plasmid DNA or mRNA, it undergoes translation within the cell, eventually forming the active RNP complex with the gRNA. Notably, many DNA repair inhibitors, typically small molecules, can also be integrated into these LNPs due to their compatibility with the lipid environment. This co-encapsulation ensures simultaneous delivery to target cells, promoting DNA cutting by Cas9 and inhibiting repair mechanisms.

Moreover, LNPs can be surface modified with specific ligands or antibodies to target unique cell types, enhancing precision. Once bound to a target cell, the LNP undergoes internalization, often through endocytosis, subsequently releasing its contents within the cell. While this composition offers a synergistic approach, especially for applications like targeted cancer therapy, it's crucial to thoroughly optimize and test the LNP's composition and properties to ensure its safety, efficiency, and specificity.

DNA repair pathways are targeted by a variety of inhibitors. Some recognized ones include PARP inhibitors: olaparib, rucaparib, niraparib, and talazoparib; ATM inhibitors: AZD0156 and KU-60019; ATR inhibitors: AZD6738 and VE-822; DNA-PK inhibitors: NU7441 and KU-57788; checkpoint kinase inhibitors: CHK1 inhibitors like MK-8776 and CHK2 inhibitors such as CCT241533; WEE1 inhibitors: adavosertib (AZD1775); RAD51 inhibitors: B02; MRE11 inhibitors: mirin and PFM39; XRCC1 inhibitors: Go6976; and HDAC inhibitors: vorinostat and romidepsin.

The gRNA, a critical component of the CRISPR-Cas9 system, binds to the Cas9 protein through a multifaceted interaction. Composed of both crRNA, which holds the sequence complementary to the target DNA, and tracrRNA, which aids in activating Cas9, these two RNA sequences are often fused to form a singular, streamlined gRNA. The Cas9 protein has distinct domains, including the REC (recognition) and NUC (nuclease) lobes. The gRNA's tracrRNA segment primarily engages with the REC lobe, especially its REC2 domain, while the crRNA segment aligns with another protein section. Upon binding, the Cas9 protein undergoes a vital conformational change, priming it to its DNA target. This gRNA-Cas9 complex then meticulously scans the DNA, searching for a sequence complementary to the gRNA, followed by a unique Protospacer Adjacent Motif (PAM) sequence. Upon successful alignment with the target DNA, the gRNA base pairs with its complementary sequence, facilitating the Cas9 protein to introduce a precise double-strand break in the DNA.

Homologous recombination (HR) and non-homologous end joining (NHEJ) are the principal pathways through which cells address DNA double-strand breaks (DSBs). HR is an intricate, high-fidelity mechanism that repairs DSBs by leveraging a homologous DNA sequence, usually the sister chromatid, as a reference template. This process, primarily active during the S and G2 phases of the cell cycle, ensures the preservation of the original DNA sequence. A key player in HR is the protein RAD51, which aids in searching for and aligning with the homologous DNA sequence. On the other hand, NHEJ directly rejoins the broken DNA ends without needing a template, making it inherently error-prone and sometimes leading to mutations at the repair site. NHEJ operates throughout the cell cycle but is incredibly dominant during the G1 phase, with proteins like the Ku70/Ku80 heterodimer and DNA-PKcs playing pivotal roles. In the context of cancer, both these mechanisms gain prominence. Some cancers exhibit a deficiency in the HR pathway, increasing their dependence on the more error prone NHEJ. Notably, mutations in genes like BRCA1 and BRCA2, linked to HR deficiency, elevate the risk of certain cancers such as breast and ovarian cancer. This HR deficiency makes such cancers more susceptible to specific treatments like PARP inhibitors. Conversely, the error-inducing nature of NHEJ can be harnessed therapeutically by introducing targeted DNA breaks, possibly via techniques like CRISPR-Cas9, and allowing NHEJ to induce potentially gene-inactivating mutations in cancer genes. HR and NHEJ are crucial in influencing cancer cells' behavior, treatment response, and resistance patterns.

DNA repair inhibitors target critical proteins in DNA damage response pathways, including homologous recombination (HR) and non-homologous end joining (NHEJ). For HR, PARP inhibitors like olaparib, rucaparib, and niraparib are the most well-known. PARP proteins play a role in the repair of single-strand DNA breaks. When PARP is inhibited, these breaks persist and, during DNA replication, can lead to double-strand breaks (DSBs). In cells with functional HR, these DSBs are repaired accurately. However, in cells with HR deficiencies, such as those with mutations in BRCA1 or BRCA2, the persistence of these DSBs can be lethal, leading to cell death. This mechanism underlies the synthetic lethality observed when combining PARP inhibitors with HR defects.

On the other hand, the NHEJ pathway, which is more error-prone and operates throughout the cell cycle, can be targeted by DNA-PK inhibitors like NU7441. DNA-PK is crucial for the NHEJ process, and its inhibition disrupts the direct ligation of DSBs, leading to the accumulation of DNA damage and potentially cell death, especially in cells that heavily rely on the NHEJ pathway.

In summary, while PARP inhibitors are designed to exploit defects in the HR pathway by causing an accumulation of DSBs that cells can't repair, DNA-PK inhibitors target the NHEJ pathway, preventing the direct rejoining of these breaks. Both inhibitors can be particularly effective in cancer cells with specific DNA repair defects or dependencies.

The ideal composition of a lipid nanoparticle (LNP) for the delivery of the gRNA-Cas9 complex, along with a DNA repair inhibitor, would prioritize effective encapsulation, stability, targeted delivery, and cellular uptake. LNPs typically consist of a mix of ionizable cationic lipids, which help in encapsulating the negatively charged RNA molecules; phospholipids, which provide structural integrity; cholesterol, which enhances membrane rigidity and stability; and polyethylene glycol (PEG)-lipids, which grant stealth properties to evade rapid clearance by the immune system. The ionizable lipid is crucial as it responds to the acidic endosomal pH, facilitating the release of the encapsulated gRNA-Cas9 complex into the cytoplasm. For the co-delivery of a DNA repair inhibitor, the LNP must also be optimized to encapsulate the specific inhibitor, a small molecule, or a peptide, ensuring its stability and release profile align with the gRNA-Cas9's activity timeline. Ideally, the LNP would also have targeting ligands on its surface to enhance specificity for cancer cells or the desired cell type, minimizing off-target effects. Crafting such an LNP requires a delicate balance of these components, and extensive in vitro and in vivo testing is imperative to ascertain efficacy, safety, and optimized delivery of both the gRNA-Cas9 complex and the DNA repair inhibitor.

Targeted delivery of the claimed composition must be administered directly into a tumor by injecting or infusing the composition to prevent off-target mutations that may arise from the exposure of healthy cells to DNA repair inhibitors. Repeated intratumoural injections with agents designed to enhance anti-tumor responses constitutes a feasible strategy to reduce the risk of systemic toxicities and achieve higher local bioactive drug concentrations.

1. A composition intended for intratumoral administration to treat cancer, comprising at least one complex of a guide RNA (gRNA), a Cas9 protein, and at least one DNA repair inhibitor, wherein the gRNA leads the complex to a site of gene mutation identified in the gRNA, to which it binds and causes the Cas9 protein to cause a double-strand break of the mutated gene, which is prevented from homologous recombination (HR) and non-homologous end joining (NHEJ) by the DNA repair inhibitor to destroy a mutated tumor cells.

2. The composition of claim 1, wherein the complex further contains a tumor-targeting molecule comprising folic acid, hyaluronic acid, polyunsaturated fatty acids, oligopeptides, or a combination thereof.

3. The composition of claim 1, wherein the sequence of gRNA includes the sequence of a mutation identified in a cancer patient or from a known structure of mutated oncogenes genes.

4. The composition of claim 3, wherein the gene sequence of mutated genes comprises (gene-UniProt Number) ABL1-P00519; AKT1-P31749; AKT2-P31751; AKT3-Q9Y243; ALK-Q9UM73; APC-096017; AR-P10275; ARID1A-014497; ATM-Q13315; ATR-Q13535; B2M-P61769; BAP1-Q92560; BARD1-Q99728; BCL10-Q92874; BCL2-P10415; BCL2L1-Q07817; BCL6-P41182; BCR-P11274; BIRC3-Q13485; BLK-P51451; BMPR1A-P36894; BRAF-P15056; BRCA1-P38398; BRCA2-P51587; BRIP1-Q9BX63; BTG1-P62324; CALR-P27797; CARD11-Q9BVK6; CASP8-Q14790; CBL-P22681; CCND1-P24385; CCND2-P30279; CCND3-P30281; CD274-Q9NZQ7; CD276-Q9BQ51; CD79A-P11912; CD79B-P40259; CDH1-P12830; CDH2-P19022; CDK4-P11802; CDK6-Q00534; CDKN1A-P38936; CDKN1B-P46527; CDKN2A-P42771; CDKN2B-Q99816; CDKN2C-Q9NRY4; CEBPA-P49715; CHEK1-O14757; CHEK2-O96017; CREBBP-Q92793; CRLF2-Q9GZX6; CSF1R-P07333; CTNNB1-P35222; CUL3-Q13618; CYLD-Q9NQC7; DAXX-Q9UER7; DDR2-Q16832; DICER1-Q9UPY3; DNMT3A-Q9Y6K1; EGFR-P00533; EML4-Q6PML9; EP300-Q09472; ERBB2-P04626; ERBB3-P21860; ERBB4-Q15303; ESR1-P03372; ETV1-P50549; ETV4-P43268; ETV5-Q00534; ETV6-P41212; EZH2-Q15910; FANCA-O15360; FANCD2-Q9BXW9; FANCE-Q9HB96; FANCF-Q9NP18; FANCG-015287; FAS-P25445; FBXW7-Q969H0; FGFR1-P11362; FGFR2-P21802; FGFR3-P22607; FLT3-P36888; FOXP1-Q9H334; GATA1-P15976; GATA2-P23769; GATA3-P23771; GNA11-P29992; GNAQ-P50148; GNAS-O95467; GNB1-P62873; GNB2L1-P62873; GPR124-Q86YT4; GRIN2A-Q12879; H3F3A-P84243; H3F3B-P84244; HIF1A-Q16665; HRAS-P01112; IDH1-075874; IDH2-O75874; IDH3A-O43837; IDH3B-Q9Y6K9; IDH3G-O75390; IGF1R-P08069; IL7R-P16871; INSR-P06213; JAK1-P23458; JAK2-060674; JAK3-P52333; KDM5A-Q9UGL1; KDM6A-015550; KDR-P35968; KEAP1-Q14145; KIT-P10721; KMT2A-Q03164; KMT2C-O14686; KMT2D-O14686; KRAS-P01116; LIFR-P42702; LRP1B-Q6UWL6; MAP2K1-Q02750; MAP2K2-P36507; MAP2K4-P45985; MAP3K1-Q13233; MAP3K4-Q9Y4K4; MAPK1-P28482; MAPK3-P27361; MAX-P61244; MCL1-Q07820; MDM2-Q00987; MED12-095243; MEN1-000255; MET-P08581; MLH1-P40692; MPL-P40238; MSH2-P43246; MSH6-P52701; MTOR-P42345; MYB-P10242; MYC-P01106; MYCL-P12524; MYCN-P04198; MYD88-Q99836; NCOA1-Q15788; NCOA2-Q15596; NCOA3-Q9Y6Q9; NF1-P21359; NF2-P35240; NOTCH1-P46531; NOTCH2-Q04721; NPM1-P06748; NRAS-P01111; NSD1-Q96L73; NTRK1-P04629; NTRK2-Q16620; NTRK3-Q16288; PAK1-Q13153; PAX3-P23760; PAX7-P23771; PBRM1-Q86U86; PDGFRA-P16234; PDGFRB-P09619; PHF6-Q8IXJ9; PIK3CA-P42336; PIK3CB-P42338; PIK3CD-O00329; PIK3CG-P48736; PIK3R1-P27986; PIK3R2-O00459; PIK3R3-O75771; PLCB1-Q01970; PMS2-P54278; POLD1-P28340; POLH-Q9Y253; PPM1D-015297; PPP2R1A-P30153; PPP2R1B-Q13362; PRDM1-O75626; PRKACA-P17612; PRKCA-P17252; PRKCB-P05771; PRKCD-Q05655; PRKDC-P78527; PTCH1-Q13635; PTEN-P60484; PTPN11-Q06124; RAD21-O60216; RAD50-Q92878; RAD51-Q06609; RAF1-P04049; RB1-P06400; RET-P07949; RHOA-P61586; RNF43-Q68DV7; ROS1-Q9NRD0; RUNX1-Q01196; SDHA-P31040; SDHB-P21912; SDHC-Q99643; SDHD-014521; SETBP1-Q9Y4C1; SF3B1-Q15788; SIRT2-Q8IXJ6; SLX4-Q8IY92; SMAD2-Q15796; SMAD3-P84022; SMAD4-Q13485; SMC1A-Q14683; SMC3-Q9NTJ3; SMO-Q99835; SPOP-O43791; SRC-P12931; SRSF2-P62807; STAG2-Q8N8T8; STAT3-P40763; STAT5B-P51692; STK11-Q15831; SUZ12-Q15022; SYNE1-Q8NF91; TBL1XR1-Q9UBK7; TCF7L2-Q9NQB0; TERT-014746; TET2-Q6N021; TFEB-P19484; TGFBR2-P37173; TLR4-O00206; TNFAIP3-Q6ZNK6; TP53-P04637; TPMT-P51580; TRAF3-O43184; TRIM24-Q13263; TRIM33-015164; TSC1-Q92574; TSC2-P49815; U2AF1-P26368; UBR5-Q7Z6Z7; USP9X-Q93008; VHL-P40337; WHSC1-060814; WRN-Q14191; WT1-P19544; XPO1-014980; YAP1-P46937; YES1-P07947; ZBTB16-P41182; ZEB1-P37275; ZFHX3-Q15911; ZMYM2-Q13263; ZMYM3-Q9Y6X9; ZMYM5-Q9UHG3; ZNF217-P14373; and ZNF750-Q6ZMV9.

5. The composition of claim 1, wherein the gRNA is a single RNA (sgRNA), transfer RNA (trRNA), or a combination thereof.

6. The composition of claim 1, wherein the DNA repair inhibitor is selected from a group of DNA repair inhibitors comprising PARP inhibitors, ATM inhibitors, ATR inhibitors, DNA-PK inhibitors, checkpoint kinase inhibitors, WEE1 inhibitors, MRE11 inhibitors, and XRCC1 inhibitors.

7. The composition of claim 6, wherein the DNA repair inhibitor comprises olaparib, rucaparib, niraparib, talazoparib, AZD7648, E7449, KU-0058948, NU5455, NU7441, U5455, ZD0156, KU-60019, AZD6738, VE-822, KU-57788, MK-8776, CCT241533, adavosertib (AZD1775), B02, mirin, PFM39, Go6976, vorinostat, and romidepsin, or a combination thereof.

8. The composition of claim 7, wherein the DNA repair inhibitor is a dual inhibitor for both HR and NHEJ.

9. The composition of claim 8, wherein the dual DNA repair inhibitor comprises of AZD7648, E7449, KU-0058948, NU5455, NU7441, Olaparib (in higher concentrations), and U5455.

10. The composition of claim 7, wherein at least one DNA inhibitor for an HR and one for NHEJ inhibition is selected.

11. The composition of claim 9, wherein only one dual DNA repair inhibitor is selected.

12. The composition of claim 1, wherein the gRNA-Cas9 complex is formulated as a lipid nanoparticle (LNP).

13. The composition of claim 1, wherein the DNA repair inhibitor is mixed with the gRNA-Case9 complex within the same LNP or in a separate LNP mixed with the LNP carrying the gRNA-Case9 complex.

14. The composition of claim 12, wherein the LNP consists of a mix of ionizable cationic lipids, which help in encapsulating the negatively charged RNA molecules; phospholipids, which provide structural integrity; cholesterol, which enhances membrane rigidity and stability; and polyethylene glycol (PEG)-lipids, which grant stealth properties to evade rapid clearance by the immune system.

15. The composition of claim 1, wherein the intratumoral administration is made by injecting or infusing the said composition in a tumor.

16. The composition of claim 1, where the said composition is used to treat breast cancer, lung cancer, melanoma, and brain tumors.


SEQUENCE LISTING (6 sequences)

SEQ ID NO: 1 - DNA, 23 bp, unidentified
  unsure 1
  source 1..23: mol_type = genomic DNA
acgtacgtacgtacgtacgtngg

SEQ ID NO: 2 - protein, 185 aa, unidentified
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKC

SEQ ID NO: 3 - protein, 82 aa, unidentified
GUUUUAGAGCUAGAAAUAGCAAGUUAAAAUAAGGCUAGUCCGUUAUCAACUUGAAAAAGUGGCACCGAGUCGGUGCUUUUUU

SEQ ID NO: 4 - DNA, 38 bp, unidentified
  source 1..38: mol_type = genomic DNA
ctggtggcgtaggcaagagtgccttgacgatacagcta

SEQ ID NO: 5 - DNA, 38 bp, unidentified
  source 1..38: mol_type = genomic DNA
ctggtggcgtaggcaagtgtgccttgacgatacagcta

SEQ ID NO: 6 - DNA, 20 bp, Homo sapiens
  source 1..20: mol_type = genomic DNA
cgtaggcaagtgtgccttga